(19) United States
(12) Patent Application Publication
(10) Pub. No.: US 2025/0213499 A1
(43) Pub. Date: Jul. 3, 2025

(54) COMPOSITION FOR PREVENTING OR TREATING ISCHEMIC STROKE, CONTAINING SOBREROL AS ACTIVE INGREDIENT

(71) Applicant: NEUROLMED CO., LTD, Anseong-si, Gyeonggi-do (KR)

(72) Inventors: Kang-Hoon JE, Anseong-si (KR); Sun Young YOON, Anseong-si (KR)

(73) Assignee: NEUROLMED CO., LTD, Anseong-si, Gyeonggi-do (KR)

(21) Appl. No.: 18/877,760

(22) PCT Filed: Aug. 19, 2021
(86) PCT No.: PCT/KR2021/011005

§ 371 Date: Feb. 7, 2025

(30) Foreign Application Priority Data
Aug. 26, 2020 (KR) 10-2020-0107790

(51) Int. Cl. A61K 31/047; A61K 9/00; A61K 9/08; A61K 9/16; A61K 9/20; A61K 9/48; A61K 47/12; A61K 47/26; A61K 47/46; A61P 9/10

(52) U.S. Cl. CPC A61K 31/047; A61K 9/0056; A61K 9/0095; A61K 9/08; A61K 9/1611; A61K 9/1623; A61K 9/2013; A61K 9/2018; A61K 9/2059; A61K 9/4858; A61K 9/4866; A61K 47/12; A61K 47/26; A61K 47/46; A61P 9/10

(57) ABSTRACT

The present invention relates to: a composition for preventing or treating a stroke and a food composition for preventing or alleviating a stroke, the compositions containing sobrerol; and the like. The present invention contains, as an active ingredient, sobrerol or a pharmaceutically acceptable salt thereof to have a brain protection effect against ischemic brain tissue damage caused by middle cerebral artery occlusion/reperfusion, and reduces the size of an infarction in the internal capsule, and thus can be effectively usable in the pharmaceutical and food fields for preventing, alleviating, or treating a stroke.

### TECHNICAL FIELD

The present invention relates to a composition for preventing and treating stroke, and more particularly, to a composition for preventing or treating stroke, a food composition for preventing or ameliorating stroke, etc., including sobrerol.

The present invention relates to a pharmaceutical composition for preventing or treating ischemic stroke, which includes sobrerol or a pharmaceutically acceptable salt thereof as an active ingredient, exhibiting a brain protective effect against ischemic brain tissue damage induced by middle cerebral artery occlusion/reperfusion, and reducing the infarction size in the internal capsule, thereby being useful in the pharmaceutical and food fields for preventing, ameliorating, or treating stroke.

### BACKGROUND ART

Stroke may be broadly divided into two types. The first is cerebral infarction, ischemic stroke, or infarct stroke, which occurs when a blood vessel is blocked and a part of the brain supplied with blood by the blood vessel is damaged. The second is cerebral hemorrhage or hemorrhagic stroke, which occurs when a cerebral blood vessel bursts and blood pools in the brain, damaging that part of the brain. In the West, the former is three times more common than the latter, and in Korea, ischemic stroke, accounting for about 85%, is known to be more common than hemorrhagic stroke.

The main cause of cerebral ischemia and cerebral infarction is a thrombus s or embolus, and the cerebral blood vessel where the thrombus or embolus is located becomes ischemic due to reduced or blocked blood flow. Neurons in the ischemic area release excitatory neurotransmitters, increasing the metabolic demands of neurons, thereby further worsening hypoxia in the ischemic area. Moreover, intracellular homeostasis is lost due to calcium influx into cells and an increase thereof, which causes damage to neurons and leads to tissue necrosis.

Cerebral infarction caused by blockage of a cerebral blood vessel is further divided into cerebral thrombosis and cerebral embolism. Cerebral thrombosis occurs when arteriosclerosis is caused by hypertension, diabetes, hyperlipidemia, etc., causing the arterial walls to thicken or harden, narrowing the blood vessels and making the inner walls of the blood vessels more susceptible to injury, resulting in blood clots and eventual blockage, significantly reducing or stopping the blood supply, leading to insufficient oxygen and nutrient supply to brain cells and impaired brain function. Cerebral embolism occurs when blood flow in the heart is abnormal due to diseases such as heart valve disease or atrial fibrillation, causing some of the blood to partially stagnate and coagulate inside the heart, forming a thrombus, which breaks off and blocks a cerebral blood vessel, resulting in cerebral infarction.

Cerebral hemorrhage is divided into primary cerebral hemorrhage, which occurs when the walls of small blood vessels cannot withstand the increased pressure inside the cerebral blood vessels due to hypertension and burst; subarachnoid hemorrhage, which occurs when a cerebral aneurysm, which is a condition in which a portion of the blood vessel wall is weak and the wall of the weak portion stretches and bulges out in the shape of a balloon, bursts for some reason; and cerebral hemorrhage, which occurs when a congenital arteriovenous malformation of the brain causes the high pressure of the artery to be directly transmitted to the vein. In addition, cerebral hemorrhage may also occur in cases where there is a predisposition to bleeding due to a blood clotting disorder.

Intracerebral hemorrhage may be caused by cerebral aneurysm, arteriovenous malformation, hypertension, trauma, infection, congenital malformation, and side effects of medications. When blood comes into contact with brain tissue due to cerebral hemorrhage, brain cell damage occurs, and rebleeding after initial aneurysm rupture, hydrocephalus, and ischemic state caused by blood loss within the blood vessels due to hemorrhage further worsen cranial nerve function.

Meanwhile, stroke (or cerebrovascular disease) is a neurological symptom that occurs when a blood vessel supplying blood to the brain is blocked or bursts, causing brain cells in the damaged area to die, resulting in physical disabilities such as loss of consciousness, communication disorders, hemiplegia, etc. Even if the patient survives, he or she will have serious aftereffects and disabilities.

Current treatment methods include drug therapy using antithrombotic agents, anticoagulants, antiplatelet agents, thrombolytic agents, and the like, but the only currently approved drug, an antithrombotic agent (tPA), is only effective within 4 to 5 hours after the onset of cerebral infarction. However, tPA may worsen hemorrhagic stroke and cause side effects such as allergic reaction, etc. Briefly, stroke is not easy to diagnose, early detection is difficult, and there is still no fundamental treatment.

The present inventors have made great efforts to study methods of treating stoke and ascertained that sobrerol has a brain neuron protective effect and is able to reduce infarction size and may thus be used to prevent, ameliorate, and treat stroke, thereby culminating in the present invention.

### DISCLOSURE

### Technical Problem

The present invention has been made keeping in mind the problems encountered in the related art, and an object of the present invention is to provide a pharmaceutical composition for preventing or treating stroke and a functional health food composition for preventing or ameliorating stroke, each including sobrerol or a pharmaceutically acceptable salt thereof as an active ingredient, thus exhibiting a brain protective effect against ischemic brain tissue damage induced by middle cerebral artery occlusion/reperfusion, and reducing the infarction size in the internal capsule, thereby being useful in the pharmaceutical and food fields for preventing, ameliorating, or treating stroke.

### Technical Solution

In order to accomplish the above object, the present invention provides a pharmaceutical composition for preventing, ameliorating, or treating stroke, including a compound represented by Chemical Formula 1 below or a pharmaceutically acceptable salt thereof as an active ingredient.

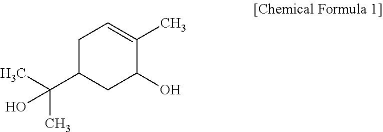

In addition, the present invention provides a food composition for preventing or ameliorating stroke, including the compound represented by Chemical Formula 1 as an active ingredient.

### Advantageous Effects

According to the present invention, a composition for preventing or treating ischemic stroke containing sobrerol as an active ingredient provides neuroprotective action.

In addition, according to the present invention, the composition for preventing or treating ischemic stroke containing sobrerol as an active ingredient not only exhibits a protective effect on brain cells, but also manifests excellent efficacy in suppressing cerebral infarction, a brain disease, by reducing the infarction size, and therefore can be useful in the pharmaceutical and food fields for preventing various types of cerebrovascular diseases and preventing, ameliorating, and treating stroke.

### DESCRIPTION OF DRAWINGS

FIG. 1 shows an ischemic stroke experimental using sobrerol as an active ingredient procedure according to the present invention;

FIG. 2 shows images of mouse slices after TTC staining to confirm the damage status of ischemic brain tissue according to the present invention; and

FIG. 3 is a graph showing results of measurement of the volume of the infarction area in mouse brain slices of individual groups according to FIG. 2.

### BEST MODE

Hereinafter, a detailed description will be given of the present invention with reference to the attached drawings.

The present inventors have studied materials effective against stroke and ascertained that the infarction size in the internal capsule of a rodent cerebral ischemia model is reduced by sobrerol, thus culminating in the present invention.

The present invention provides a pharmaceutical composition for preventing or treating stroke, including a compound represented by Chemical Formula 1 below or a pharmaceutically acceptable salt thereof as an active ingredient.

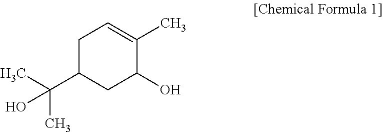

“Sobrerol (5-(2-hydroxypropan-2-yl)-2-methylcyclohex-2-en-1-ol)” represented by Chemical Formula 1 is a terpene alcohol with the chemical formula of C₁₀H₁₈O₂, in which “terpene” is a flammable unsaturated hydrocarbon and is a generic term for carbohydrates and derivatives thereof such as alcohols, aldehydes, ketones, etc., with the chemical formula of (C₆H₈)ₙ.

Regarding specific pharmacological activity of the compound described above, it has not been revealed whether it directly has a preventive and therapeutic effect on stroke.

A method of obtaining sobrerol is not particularly limited in the present invention, and sobrerol may be chemically synthesized by a method known in the art, or a commercially available material may be used.

The sobrerol of the present invention may be present not only in a solvated form but also in an unsolvated form. The sobrerol of the present invention may be present in crystalline or amorphous form, and all such physical forms are within the scope of the present invention.

The pharmaceutical composition according to the present invention may include not only the compound represented by Chemical Formula 1 but also a pharmaceutically acceptable salt thereof. As used herein, the term “pharmaceutically acceptable salt” refers to a material in the form of a salt in which the compound is coupled with another material, and which may exhibit pharmaceutically similar activity.

The types of pharmaceutically acceptable salts include, but are not limited to, inorganic acid salts such as hydrochlorides, hydrobromides, phosphates, or sulfates, and organic acid salts such as carboxylates or sulfonates. Also, the types of carboxylates include, but are not limited to, acetate, maleate, fumarate, malate, citrate, tartrate, lactate, or benzoate. Also, the types of sulfonates include, but are not limited to, methanesulfonate, ethanesulfonate, benzenesulfonate, toluene sulfonate, or naphthalenedisulfonate.

The pharmaceutical composition according to the present invention may serve to promote recovery of motor function impaired due to stroke or reduce infarction size.

In a specific embodiment of the present invention, when the compound of Chemical Formula 1 according to the present invention was administered to a stroke animal model, the infarction size was confirmed to decrease compared to the group not administered a stroke drug.

Based on the above results, it was confirmed that the composition including the compound of Chemical Formula 1 according to the present invention has an excellent effect on the prevention or treatment of stroke.

As used herein, the term “prevention” refers to any action of inhibiting or delaying the onset of stroke by administering a composition including the compound of Chemical Formula 1 according to the present invention. The term “treatment” used herein refers to any action of alleviating or beneficially changing symptoms of the disease by administering a composition including the compound of Chemical Formula 1 according to the present invention.

The pharmaceutical dosage form of the composition for preventing or treating stroke according to the present invention may also be used in the form of a pharmaceutically acceptable salt thereof, and the composition may also be used alone or in combination with other pharmaceutically active compounds as well as in suitable aggregation therewith.

The composition for preventing or treating stroke according to the present invention may further include a pharmaceutically acceptable carrier.

The composition for preventing or treating stroke according to the present invention may be prepared into a pharmaceutical formulation using methods well known in the art so as to provide rapid, sustained, or delayed release of the active ingredient after administration to a mammal.

Therefore, the composition for preventing or treating stroke according to the present invention may be prepared and used in the form of oral formulations such as powders, granules, tablets, capsules, suspensions, emulsions, syrups, aerosols, etc., external preparations, suppositories, and sterile injectable solutions by typical methods, and may further include an appropriate carrier, excipient, and diluent typically used in the preparation of compositions.

Examples of the carrier that may be included in the composition of the present invention include, but are not limited to, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline pyrrolidone, water, methyl cellulose, polyvinyl hydroxybenzoate, propyl hydroxybenzoate, talc, magnesium stearate, and mineral oil. The formulations are prepared using typical diluents or excipients such as fillers, extenders, binders, wetting agents, disintegrants, surfactants, etc.

Solid preparations for oral administration include tablets, pills, powders, granules, capsules, etc., and these solid preparations are formulated by mixing the compound with at least one excipient, such as starch, calcium carbonate, sucrose or lactose, gelatin, etc. In addition to simple excipients, lubricants such as magnesium stearate and talc are also used.

Formulations for parenteral administration include sterile aqueous solutions, non-aqueous solutions, suspensions, emulsions, lyophilized preparations, and suppositories. Non-aqueous solutions and suspensions include propylene glycol, polyethylene glycol, vegetable oils such as olive oil, injectable esters such as ethyl oleate, and the like. As a base for suppositories, Witepsol, Macrogol, Tween 61, cacao butter, laurin oil, glycerogelatin, etc. may be used.

The pharmaceutical composition according to the present invention may be administered as an individual therapeutic agent or in combination with another therapeutic agent, and may be administered sequentially or simultaneously with a conventional therapeutic agent. Also, the composition may be administered single or multiple times. Considering all of the above factors, it is important to administer an amount capable of achieving the maximum effect with the minimum amount without causing side effects, which may be easily determined by a person skilled in the art.

The mode of administration of the pharmaceutical composition according to the present invention is not particularly limited, and may follow a mode commonly used in the relevant technical field. As a non-limiting example of the mode of administration, the composition may be administered by oral administration or parenteral administration. The pharmaceutical composition according to the present invention may be prepared in various dosage forms depending on the desired administration mode.

The frequency of administration of the composition according to the present invention is not particularly limited, but the composition may be administered once a day or administered several times in divided doses.

A typical daily dosage of the composition including the compound of Chemical Formula 1 according to the present invention as an active ingredient may be 1 to 1,000 mg/kg, particularly 11 to 100 mg/kg, and may be administered once or in several divided doses.

Another aspect of the present invention provides a food composition for preventing or ameliorating stroke, including the compound represented by Chemical Formula 1 as an active ingredient.

The food composition for preventing or ameliorating stroke according to the present invention is provided in forms such as pills, powders, granules, infusions, tablets, capsules, or liquids, and examples of foods to which the composition of the present invention may be added include various foods, such as beverages, gum, tea, vitamin complexes, functional health foods, and the like.

There is no particular limitation on other ingredients in addition to containing the composition or active ingredient thereof, or a physiologically acceptable salt thereof as an essential ingredient that may be included in the food composition for preventing or ameliorating stroke according to the present invention, and various herbal extracts, food additives, or natural carbohydrates may be contained as additional ingredients like typical foods.

A better understanding of the present invention may be obtained through the following examples.

However, the following examples are merely set forth to illustrate the present invention, and are not to be construed as limiting the scope of the present invention.

### 1. Examples: Experimental Method

### (1) Experimental Example 1. Preparation of Drug for Administration

### Preparation of sobrerol (5-(2-hydroxypropan-2-yl)-2-methylcyclohex-2-en-1-ol)

Sobrerol (5-(2-hydroxypropan-2-yl)-2-methylcyclohex-2-en-1-ol) used in this experiment was purchased from Merck (247774) and dissolved in saline for use in the experiment.

### (2) Experimental Example 2. Creation of Rodent Cerebral Ischemia Model

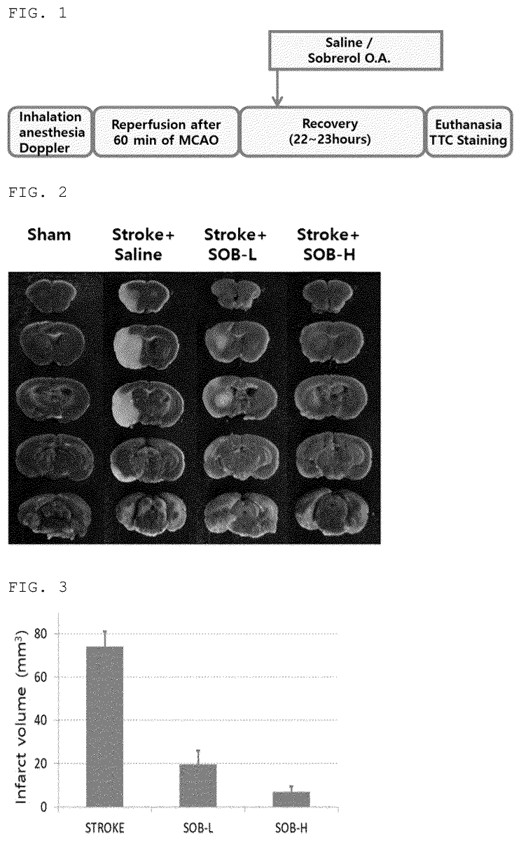

12- to 13-week-old male mice (C57/BL6) were used in the experiment. The mice were divided into four groups of five each (three in the Sham group): Sham (surgical control group), stroke surgery group (Stroke), stroke+sobrerol-L (administration of 10 mg/kg of SOB-L), and stroke+sobrerol-H (administration of 40 mg/kg of SOB-H). According to the schematic view shown in FIG. 1, middle cerebral artery occlusion and reperfusion operation (MCAO/R) was performed to induce focal cerebral ischemia. After MCAO/R surgery, sobrerol was orally administered at concentrations of 10 mg/kg and 40 mg/kg. In the Sham and Stroke control groups, saline was orally administered.

Neurological deficit tests were performed 22-23 hours after MCAO/R (n=5). After re-anesthesia, cardiac reperfusion in mice was carried out with PBS solution, and the brains were immediately removed and stained with 2% TTC (2,3,5-triphenyl-tetrazolium chloride) solution.

### (3) Experimental Example 3. Measurement of Volume of Cerebral Infarction Area

TTC-stained slices were obtained from the mice in four groups, particularly Sham (surgical control group), stroke surgery group (Stroke), stroke+sobrerol-L (administration of 10 mg/kg of SOB-L), and stroke+sobrerol-H (administration of 40 mg/kg of SOB-H), and the volume of the cerebral infarction area was measured. Quantification was measured using ImageJ.

### (4) Experimental Results

As shows in FIG. 2, brain slices of the four groups were obtained by slicing at 2 mm intervals from the frontal pole of the brain, and the volume of cerebral infarction was measured. The white area represents the infarction area and the red area represents the undamaged area. It was significantly confirmed that the mouse tissue in the stroke+sobrerol-H group was undamaged as represented by the red area compared to the stroke+sobrerol-L group, the stroke surgery group (Stroke), and the surgical control group (Sham). Briefly, it was significantly confirmed that the group that was orally administered sobrerol had red undamaged area without cerebral infarction area compared to the other groups.

In addition, as shown in the quantitative graph of FIG. 3, for the volume of the infarction area in the brain slices of each group, the cerebral infarction volume due to focal cerebral ischemia was significantly reduced in the groups that received oral administration of both concentrations of sobrerol (*** p<0.001).

Below, formulation examples of the composition containing the powder according to the present invention are described, but are not intended to limit the present invention and are merely set forth to specifically illustrate the present invention.

### Formulation Example 1. Preparation of Powder

500 mg of sobrerol

100 mg of lactose

10 mg of talc

A powder is prepared by mixing these ingredients followed by loading into a sealed bag.

### Formulation Example 2. Preparation of Tablet

300 mg of sobrerol

100 mg of corn starch

100 mg of lactose

2 mg of magnesium stearate

A tablet is prepared by mixing these ingredients followed by pressing according to a typical tablet preparation method.

### Formulation Example 3. Preparation of Capsule

200 mg of sobrerol

3 mg of crystalline cellulose

14.8 mg of lactose

0.2 mg of magnesium stearate

A capsule is prepared by mixing these ingredients followed by loading into a gelatin capsule according to a typical capsule preparation method.

### Formulation Example 4. Preparation of Injectable Solution

600 mg of sobrerol

180 mg of mannitol

2974 mg of sterile distilled water for injection

26 mg of Na₂HPO₄·12H₂O

An injectable solution is prepared using these ingredients in the amounts described above per ampoule according to a typical injectable solution preparation method.

### Formulation Example 5. Preparation of Liquid Formulation

4 g of sobrerol

10 g of isomerized sugar

5 g of mannitol

### Appropriate Amount of Purified Water

A liquid formulation is prepared by dissolving individual ingredients in purified water, adding an appropriate amount of lemon flavor thereto, mixing the ingredients, adding purified water so that the total amount is adjusted to 100 g, and then filling a brown bottle with the resulting liquid followed by sterilization according to a typical liquid preparation method.

### Formulation Example 6. Preparation of Granule

1,000 mg of sobrerol

### Appropriate Amount of Vitamin Mixture

70 μg of vitamin A acetate

1.0 mg of vitamin E

0.13 mg of vitamin B1

0.15 mg of vitamin B2

0.5 mg of vitamin B6

0.2 μg of vitamin B12

10 mg of vitamin C

10 μg of biotin

1.7 mg of nicotinamide

50 μg of folic acid

0.5 mg of calcium pantothenate

### Appropriate Amount of Mineral Mixture

1.75 mg of ferrous sulfate

0.82 mg of zinc oxide

25.3 mg of magnesium carbonate

15 mg of potassium phosphate monobasic

55 mg of calcium phosphate dibasic

90 mg of potassium citrate

100 mg of calcium carbonate

24.8 mg of magnesium chloride

The composition ratios of the vitamin and mineral mixtures were according to a preferred example of mixing ingredients relatively suitable for granules, but the mixing ratio may be arbitrarily modified, and a granule may be prepared by mixing the ingredients according to a typical granule preparation method, and may then be used to prepare a functional health food composition according to a typical method.

### Formulation Example 7. Preparation of Functional Beverage

1,000 mg of sobrerol

1,000 mg of citric acid

100 g of oligosaccharide

2 g of plum concentrate

1 g of taurine

Appropriate amount of purified water to make a total of 900 ml

According to a typical functional beverage preparation method, these ingredients are mixed and then heated with stirring at 85° C. for about 1 hour, after which the resulting solution is filtered, placed in a 2 L sterile container, sealed, sterilized, stored in a refrigerator, and then used to prepare a functional beverage composition according to the present invention.

The composition ratio was set according to a preferred example of mixing ingredients relatively suitable for favorite beverages, but the mixing ratio may be arbitrarily modified depending on regional and national preferences such as demand class, demand country, end use, and the like.

Although the present invention has been described in detail through specific embodiments thereof, the terms specifically used herein are only used for the purpose of describing the present invention and are not used to limit the meaning or the scope of the present invention described in the claims. Therefore, those skilled in the art will recognize that various modifications of these embodiments and other equivalent embodiments are possible.

1. A method of treating stroke in a subject in need thereof, comprising administering an effective amount of a pharmaceutical composition comprising a compound represented by Chemical Formula 1 below or a pharmaceutically acceptable salt thereof as an active ingredient, to the subject

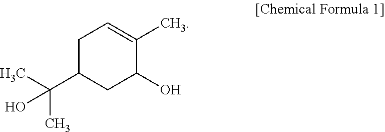

2. The method according to claim 1, wherein the stroke is an ischemic stroke.

3. The method according to claim 1, wherein the subject has an infarction in an internal capsule and the administering of the pharmaceutical composition reduces a size of the infarction in an internal capsule.

4. A method of ameliorating stroke in a subject in need thereof, comprising administering an effective amount of a food composition comprising a compound represented by Chemical Formula 1 below as an active ingredient, to the subject: [chemical structure]
